(19) United States
(12) Patent Application Publication
(10) Pub. No.: US 2026/0131151 A1
(43) Pub. Date: May 14, 2026

(54) WAVELET-BASED NEUROMODULATION SYSTEM WITH INTERACTIVE WAVEFORM EDITING

(71) Applicant: AJ Robotics LLC, Agua Dulce, CA (US)

(72) Inventors: Dongchul Lee, Agua Dulce, CA (US); Emarit Ranu, Fort Collins, CO (US)

(73) Assignee: AJ Robotics LLC, Agua Dulce, CA (US)

(21) Appl. No.: 19/383,046

(22) Filed: Nov. 7, 2025

Related U.S. Application Data
(60) Provisional application No. 63/717,988, filed on Nov. 8, 2024.

(51) Int. Cl. A61N 1/372; A61N 1/36

(52) U.S. Cl. CPC A61N 1/37247; A61N 1/36135; A61N 1/36192; A61N 1/36196

(57) ABSTRACT

A computing system for programming a stimulation waveform for delivery by an electrical stimulation system is disclosed. The computing system includes a user interface comprising a waveform editor configured to enable user-defined editing of one or more wavelets derived from a mother wavelet to generate a wavelet pulse. The wavelet pulse defines a morphology of the stimulation waveform. The computing system further includes a communication interface configured to transmit the wavelet pulse to a pulse generator of the electrical stimulation system configured to electrically modulate electrically-activatable tissue according to the wavelet pulse.

### CROSS-REFERENCE TO RELATED APPLICATIONS

This application claims the benefit of U.S. Provisional Application No. 63/717,988, filed Nov. 8, 2024, which is incorporated by reference herein.

### FIELD

The present disclosure concerns systems, devices, and methods for programming and delivering electrical stimulation waveforms based on wavelets, including the generation and modulation of wavelet pulses for application to electrically-activatable tissue, such as neural tissue.

### BACKGROUND

Neuromodulation and related electrical stimulation therapies are used to influence the activity of electrically-activatable tissue (also referred to as electrically excitable tissue), such as neural or muscular structures, for therapeutic benefit. Conventional stimulation systems typically rely on simple, repetitive pulse waveforms that may not adequately replicate physiological signaling patterns or selectively target specific tissue responses. As a result, their effectiveness can be limited, and unwanted side effects may occur. Thus, there is room for improvement in the design and programming of stimulation waveforms for electrical modulation of excitable tissue.

### SUMMARY

Described herein are systems and methods for generating and programming stimulation waveforms based on wavelets and wavelet-derived pulse structures, which provide flexible control of waveform morphology, timing, and spectral characteristics to achieve targeted modulation of electrically excitable tissue. While examples throughout this disclosure may refer to neuromodulation or stimulation of neural tissue, the disclosed technologies are more broadly applicable to electrical modulation of electrically-activatable tissues, including but not limited to neural, muscular, and other excitable tissues. Accordingly, references to neuromodulation should be understood as encompassing electrically-activatable tissue modulation more generally.

Certain aspects of the disclosure concern a computing system for programming a stimulation waveform for delivery by an electrical stimulation system. The computing system includes a user interface comprising a waveform editor configured to enable user-defined editing of one or more wavelets derived from a mother wavelet to generate a wavelet pulse. The wavelet pulse defines a morphology of the stimulation waveform. The computing system further includes a communication interface configured to transmit the wavelet pulse to a pulse generator of the electrical stimulation system configured to electrically modulate electrically-activatable tissue according to the wavelet pulse.

Certain aspects of the disclosure concern a computer-implemented method for programming a stimulation waveform for delivery by an electrical stimulation system. The method includes providing, by a computing system, a user interface; receiving, through the user interface, user input defining a mother wavelet; and generating, by the computing system, a wavelet pulse defining a morphology of the stimulation waveform. The wavelet pulse is synthesized from one or more wavelets derived from the mother wavelet.

Certain aspects of the disclosure concern an electrical stimulation system including a programming device comprising a waveform editor configured to generate a wavelet pulse defining a morphology of a finite-length stimulation waveform. The wavelet pulse is synthesized from one or more wavelets derived from a mother wavelet. The electrical stimulation system further includes a pulse generator in communication with the programming device and configured to electrically modulate electrically-activatable tissue according to the wavelet pulse.

Certain aspects of the disclosure concern an electrical stimulation system including a programming device configured to program a wavelet pulse, and a pulse generator configured to deliver electrical stimulation to electrically-activatable tissue according to the wavelet pulse programmed by the programming device. The wavelet pulse comprises one or more wavelets derived from a mother wavelet. The mother wavelet has a continuous first derivative and a time-varying instantaneous frequency.

Certain aspects of the disclosure concern a method for electrical stimulation. The method includes generating, by a programming device, a wavelet pulse comprising one or more wavelets derived from a mother wavelet. The mother wavelet has a continuous first derivative and a time-varying instantaneous frequency. The method further includes delivering, by a pulse generator, electrical stimulation to electrically-activatable tissue according to the wavelet pulse.

Certain aspects of the disclosure concern a programming device for electrical stimulation. The programming device includes a user interface configured to enable selection of a wavelet pulse comprising one or more wavelets derived from a mother wavelet. The mother wavelet has a continuous first derivative and a time-varying instantaneous frequency. The programming device further includes a communication unit configured to transmit the wavelet pulse to a pulse generator for delivery of electrical stimulation to electrically-activatable tissue according to the wavelet pulse.

Although primarily described in the context of programming stimulation waveforms for neuromodulation systems, it should be understood that the systems and methods disclosed herein may also be applied to other types of devices that utilize configurable pulse parameters for modulating electrically-activatable tissue. For example, the disclosed waveform generation and programming techniques may be used to define and deliver customized stimulation patterns for functional electrical stimulation devices used in rehabilitation therapy, transcutaneous electrical nerve stimulation devices used for pain management, implantable cardiac pacemakers and/or cardioverter-defibrillators, and other stimulation systems. More generally, the disclosed technologies may be adapted for any device that benefits from precise control of stimulation pulse morphology, timing, or spectral content.

Although the term “stimulation” is used throughout this disclosure for convenience, it should be understood that the disclosed wavelet pulse can also be applied to electrically-activatable tissue to reduce or inhibit activity, for example, by inducing hyperpolarization or otherwise blocking neural conduction. Accordingly, references to electrically modulating electrically-activatable tissue encompass both activation and deactivation of such tissue, as described herein.

The foregoing and other features and advantages of the disclosed technologies will become more apparent from the following detailed description, which proceeds with reference to the accompanying figures.

### BRIEF DESCRIPTION OF THE DRAWINGS

FIG. 1 is a schematic view of an example electrical stimulation system including a paddle lead coupled to a control module.

FIG. 2 is a schematic view of an example electrical stimulation system including a percutaneous lead coupled to a control module.

FIG. 3A is a schematic view of an example control module configured to couple to an elongated device.

FIG. 3B is a schematic view of an example lead extension configured to couple an elongated device to a control module.

FIG. 4 is a schematic overview of example components of a stimulation system, including an electronic subassembly within a control module.

FIG. 5 is a block diagram of an example programming device for a neuromodulation system, according to one example.

FIG. 6 depicts some example mother wavelets.

FIG. 7 depicts some example wavelet pulses generated based on some mother wavelets.

FIG. 8 is a flowchart illustrating an example overall method for implementing wavelet-based electrical stimulation.

FIG. 9 is a flowchart illustrating an example overall method for programming a stimulation waveform for delivery by an electrical stimulation system.

FIG. 10 depicts an example graphical user interface including a waveform editor configured to edit a wavelet pulse.

FIG. 11 depicts an example wavelet pulse and its time-frequency representation.

FIG. 12 is a block diagram of an example computing system in which described technologies can be implemented.

### DETAILED DESCRIPTION

### Overview of Neuromodulation Systems

Neuromodulation systems are medical technologies that deliver electrical modulation to electrically-activatable tissues of the body, such as the neural tissue and/or muscle tissue, to alleviate symptoms of various conditions, such as chronic pain, movement disorders, neurological dysfunctions, etc. These systems typically include a control module (e.g., a pulse generator), one or more leads with electrodes for delivering stimulation, and programming software that allows clinicians and/or patients to configure the stimulation parameters. The effectiveness of neuromodulation therapy depends in part on the proper selection and adjustment of stimulation parameters such as pulse amplitude, pulse width, frequency, phase configuration, and waveform shape, etc.

Conventional neuromodulation systems commonly employ rectangular pulse waveforms that are repeated at a fixed rate to produce continuous or patterned stimulation. Although such rectangular pulse shapes are straightforward to implement and control, they are often limited in their ability to emulate natural physiological signaling patterns. These waveforms can activate a broad population of neural fibers simultaneously, which may lead to non-specific effects, habituation, and/or stimulation inefficiency. The rigid structure of rectangular pulses also restricts the flexibility needed to tune the spectral content of stimulation for targeted neural pathways or dynamic tissue responses.

In addition, most existing systems rely primarily on adjusting global stimulation parameters such as amplitude or frequency. These limited programming options of stimulation parameters may fail to address the complexity and variability of individual patient responses or to optimize therapeutic outcomes. As a result, clinicians often must rely on empirical parameter adjustment rather than on direct waveform design or fine-grained control of the temporal and spectral morphology of stimulation.

The technology described herein provides an adaptive framework for generating and programming stimulation waveforms using configurable, wavelet-based pulse structures that allow fine control over waveform morphology, timing, and spectral content. As described more fully below, this solution enables the creation of stimulation patterns that can be tailored to individual physiology and/or therapy goals, offering enhanced selectivity and flexibility compared to conventional pulse-based neuromodulation systems.

### Example Neuromodulation Systems

The present disclosure relates to electrical stimulation systems, and more particularly to neuromodulation systems that deliver therapeutic electrical stimulation (e.g., pulses) to neural, muscular, and/or other excitable tissue (also referred to as “target tissue”) in the body. Although the examples provided herein primarily depict implantable neuromodulation systems, the disclosed technologies are also applicable to external or wearable devices. The stimulation pulses delivered by such systems can be programmed using the waveform editing technologies described herein.

Suitable neuromodulation systems can include at least one lead having one or more electrodes disposed along its distal end and one or more terminals disposed along a proximal end. Leads may take the form of paddle leads, percutaneous leads, or cuff leads, and may be used with or without extensions, adaptors, or splitters. In some cases, a stimulation system can include an implantable microstimulator, where stimulation is delivered via electrodes integrated into the device housing.

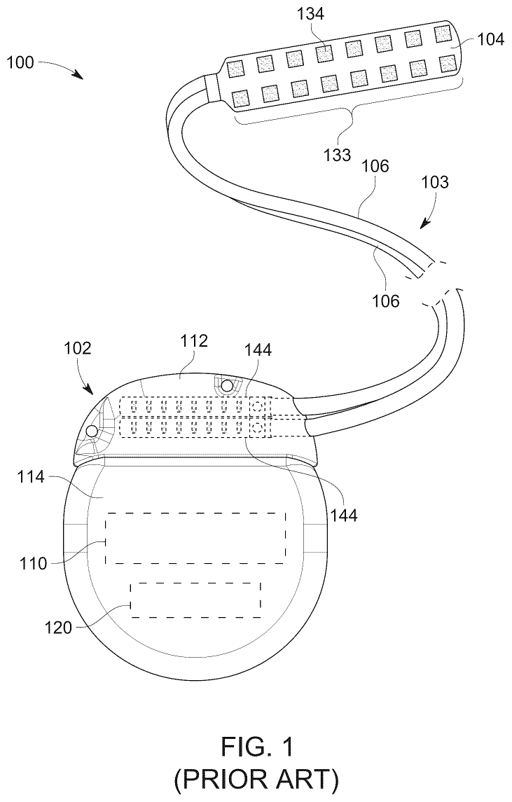

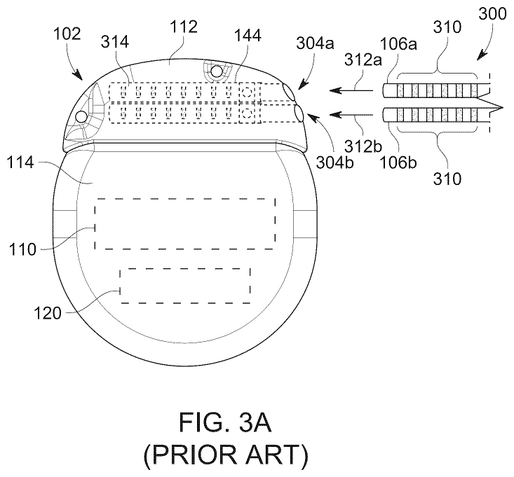

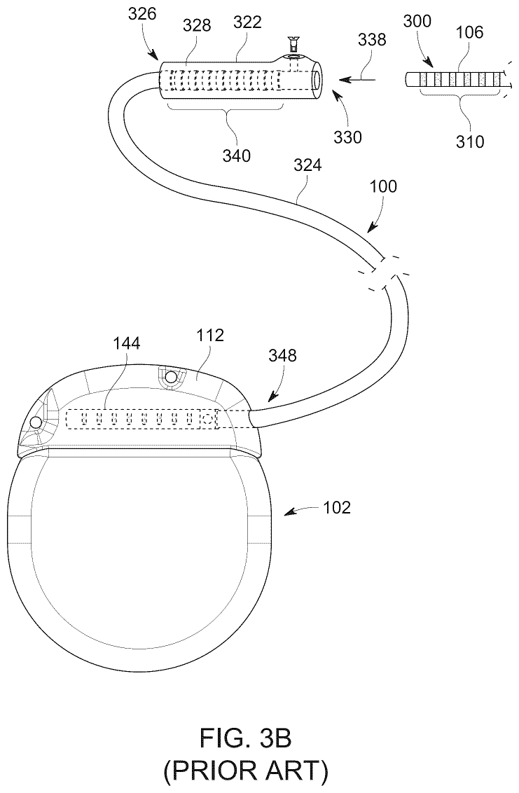

FIG. 1 schematically illustrates one example of an electrical stimulation system 100. The electrical stimulation system 100 includes a control module 102 (e.g., a stimulator or pulse generator) and a lead 103 that can be coupled to the control module 102. The lead 103 includes a paddle body 104 and one or more lead bodies 106. In the illustrated example, the lead 103 has two lead bodies 106, although any suitable number may be used, such as one, two, three, four, five, six, seven, eight, or more. An electrode array 133, including electrodes such as electrode 134, can be disposed on the paddle body 104. An array of terminals (e.g., 310 in FIGS. 3A and 3B) can be disposed along each of the one or more lead bodies 106.

It will be appreciated that the electrical stimulation system 100 may include additional, fewer, or alternative components, and may be implemented in a variety of configurations beyond those specifically illustrated. For example, instead of a paddle body, the electrodes may be arranged in an array at or near the distal end of a lead body to form a percutaneous lead. In some embodiments, a paddle lead may include only a single electrode, and similarly, a percutaneous lead may include just one electrode. Additionally, the control module itself may include one or more electrodes in certain configurations.

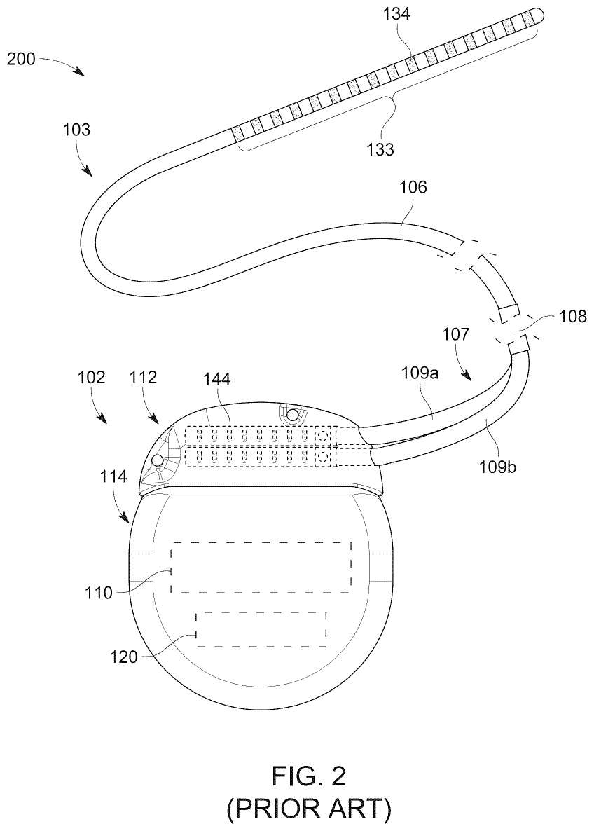

FIG. 2 schematically illustrates another embodiment of the electrical stimulation system 200, in which the lead 103 is configured as a percutaneous lead. In this example, electrodes 134 are shown disposed along one or more lead bodies 106. In at least some implementations, the lead 103 can be isodiametric along the longitudinal length of the lead body 106.

The lead 103 can be coupled to the control module 102 in any suitable manner. In the example shown in FIG. 1, the lead 103 is coupled directly to the control module 102. In other examples, the lead 103 may be connected to the control module 102 via one or more intermediate devices, such as a lead extension 324 illustrated in FIG. 3B. One or more lead extensions may be used to increase the distance between the lead 103 and the control module 102. Additionally, other intermediate components, such as splitters, adaptors, or combinations thereof, may be used in place of, or in addition to, lead extensions. Where multiple elongated devices are positioned between the lead 103 and the control module 102, the intermediate devices may be arranged in any configuration suitable for the specific clinical or system requirements.

In FIG. 2, the electrical stimulation system 200 includes a splitter 107 configured to facilitate coupling of the lead 103 to the control module 102. The splitter 107 includes a splitter connector 108 that connects to the proximal end of the lead 103, and one or more splitter tails 109a and 109b that are configured to couple to the control module 102, or alternatively to another splitter, lead extension, adaptor, or similar component.

With reference to FIGS. 1 and 2, the control module 102 typically includes a connector housing 112 and a sealed electronics housing 114. The electronics housing 114 contains an electronic subassembly 110 and, in some embodiments, an optional power source 120. A control module connector 144 can be disposed within the connector housing 112 and be configured to establish an electrical connection between the lead 103 and the electronic subassembly 110 of the control module 102.

The disclosed electrical stimulation systems, or components thereof (e.g., the paddle body 104, one or more lead bodies 106, and the control module 102) can be implanted within a patient's body. The disclosed electrical stimulation systems may be used in a variety of therapeutic applications, including but not limited to deep brain stimulation, neural stimulation, spinal cord stimulation, muscle stimulation, and similar neuromodulation therapies. In some examples, the electrical stimulation systems disclosed herein can be used for external or transcutaneous stimulation, in which some or all components remain outside the body and deliver electrical energy through the skin via surface electrodes for applications such as pain management, rehabilitation, etc.

The electrodes 134 may be formed from any conductive, biocompatible material. Suitable materials include metals, metal alloys, conductive polymers, conductive carbon, and combinations thereof. In some examples, one or more electrodes 134 can be made from materials such as platinum, platinum-iridium, palladium, palladium-rhodium, or titanium.

Any suitable number of electrodes 134 may be disposed on the lead, including, for example, one, two, three, four, five, six, seven, eight, nine, ten, eleven, twelve, fourteen, sixteen, twenty-four, thirty-two, or more. For paddle leads, the electrodes 134 can be arranged on the paddle body 104 in a variety of configurations. For example, in FIG. 1, the electrodes are arranged in two columns, with each column containing eight electrodes.

The electrodes on the paddle body 104 (or on one or more lead bodies 106) can be embedded in or separated by a non-conductive, biocompatible material such as silicone, polyurethane, polyetheretherketone (PEEK), epoxy, or combinations thereof. The lead bodies 106 and, where applicable, the paddle body 104 may be formed into the desired shape using manufacturing processes such as molding (including injection molding), casting, or similar techniques. The non-conductive material can extend along each lead body 106 from its distal end to its proximal end.

For paddle leads, the non-conductive material can extend from the paddle body 104 to the proximal end of each lead body 106. The biocompatible, non-conductive material used in the paddle body 104 and the lead bodies 106 may be the same or different. Additionally, the paddle body 104 and the lead bodies 106 may be formed as a single, unitary structure or as separate components that are permanently or detachably coupled together.

Terminals (e.g., 310 in FIGS. 3A and 3B) can be disposed along the proximal end of one or more lead bodies 106 (e.g., 106a, 106b) of the electrical stimulation system 100 or 200, as well as on any intermediate components such as splitters, lead extensions, or adaptors. These terminals can provide electrical connections to corresponding connector contacts (e.g., 314 in FIG. 3A), which are housed within connectors (e.g., 144 in FIGS. 1-3B; 322 in FIG. 3B) located on the control module 102 or on associated intermediate devices. Electrically conductive wires, cables, or similar structures can extend from the terminals to the electrodes 134. In some examples, one or more electrodes 134 can be electrically coupled to each terminal. In some examples, each terminal is only connected to a single electrode 134.

The electrically conductive wires (or “conductors”) may be embedded within the non-conductive material of the lead body 106 or housed in one or more lumens extending along the length of the lead body. In some examples, each conductor is routed through a dedicated lumen, while in others, multiple conductors can share a common lumen. One or more lumens may open near the proximal end of the lead body 106 to allow insertion of a stylet, aiding in placement of the lead within the patient's body. Additionally, one or more lumens may open near the distal end to permit infusion of drugs or medication at the implantation site. In certain examples, the lumens can be flushed continuously or periodically with saline, epidural fluid, or similar substances. Some lumens may also be configured to be permanently or removably sealed (e.g., at the distal end).

FIG. 3A is a schematic side view of one embodiment of a proximal end of one or more elongated devices 300, configured to couple with the control module connector 144. The elongated devices 300 may include, for example, one or more lead bodies 106 shown in FIG. 1, as well as intermediate components such as a splitter, the lead extension 324 shown in FIG. 3B, an adaptor, or combinations thereof.

In some examples, the control module connector 144 defines at least one port for receiving a proximal end of the elongated device 300, as indicated by directional arrows 312a and 312b. In FIG. 3A, the connector housing 112 is shown with two ports, 304a and 304b. However, the connector housing 112 may define any suitable number of ports, including one, two, three, four, five, six, seven, eight, or more.

Each port 304a and 304b of the control module connector 144 can include a plurality of connector contacts, such as connector contact 314. When the elongated device 300 is inserted into these ports, the connector contacts 314 can align with a corresponding plurality of terminals 310 disposed along the proximal ends of the elongated device(s) 300. This alignment can establish an electrical connection between the control module 102 and the electrodes (e.g., electrodes 134 in FIG. 1) located on the paddle body 104 of lead 103.

FIG. 3B is a schematic side view of another embodiment of the electrical stimulation system 100 or 200. In this example, the system includes a lead extension 324 configured to couple one or more elongated devices 300, such as a lead body 106 (FIGS. 1 and 2), the splitter 107 (FIG. 2), an adaptor, another lead extension, or combinations thereof, to the control module 102. In FIG. 3B, the lead extension 324 is shown connected to a single port of the control module connector 144 and arranged to couple with a single elongated device 300. In other examples, the lead extension 324 may be configured to connect to multiple ports on the control module connector 144, to receive multiple elongated devices 300, or both.

In some examples, a lead extension connector 322 can be positioned on the lead extension 324. In FIG. 3B, the connector 322 is shown at the distal end 326 of the lead extension. The connector 322 includes a housing 328 that defines at least one port 330 for receiving terminals 310 of the elongated device 300, as indicated by directional arrow 338. Within the housing 328, a plurality of connector contacts, such as contacts 340, are provided. When the elongated device 300 is inserted into port 330, the connector contacts 340 can align with the terminals 310 to electrically couple the lead extension 324 to the electrodes (e.g., electrodes 134 of FIGS. 1 and 2) disposed along lead 103.

In some examples, the proximal end of the lead extension 324 can be configured similarly to the proximal end of the lead 103 (or another elongated device 300). The lead extension 324 may include a plurality of electrically conductive wires that extend from the connector contacts 340 to a proximal end 348, opposite the distal end 326. These conductive wires may be electrically coupled to a plurality of terminals disposed along the proximal end 348. In certain implementations, the proximal end 348 can be configured for insertion into a connector of another lead extension or intermediate device. In other examples, such as the example shown in FIG. 3B, the proximal end 348 can be configured for direct insertion into the control module connector 144.

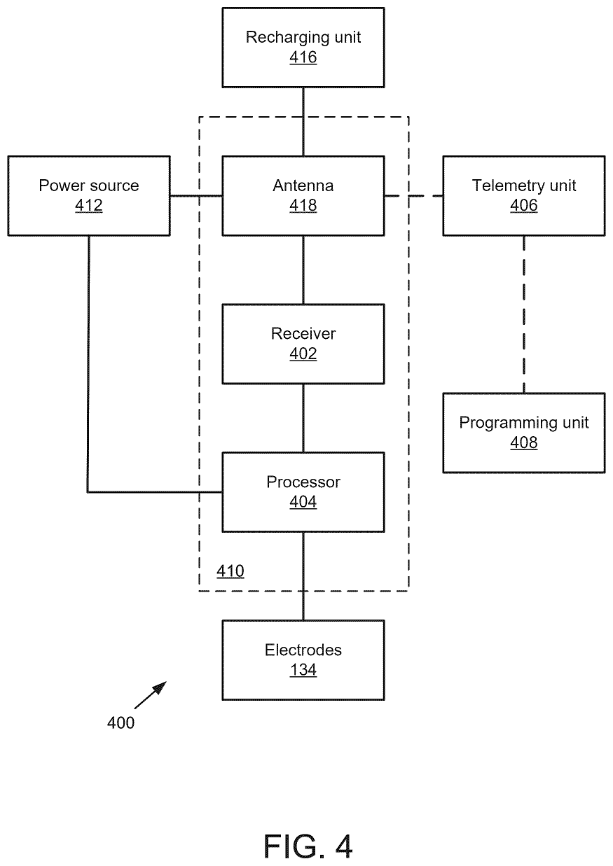

FIG. 4 is a schematic overview of components of an example electrical stimulation system 400, including an electronic subassembly 410 housed within a control module. It should be understood that the electrical stimulation system 400 may include additional, fewer, or alternative components, and can be implemented in a variety of configurations beyond those specifically illustrated.

Various components of the electrical stimulation system, such as a power source 412, an antenna 418, a receiver 402, and a processor 404, can be mounted on one or more circuit boards or similar carriers within a sealed housing of an implantable pulse generator or control module, or within the housing of a microstimulator. The power source 412 may be any suitable type, including primary or rechargeable batteries. Other possible power sources include supercapacitors, nuclear or atomic batteries, mechanical resonators, infrared collectors, thermally powered generators, flexural energy harvesters, bioenergy sources, fuel cells, bioelectric cells, and osmotic pressure pumps.

Alternatively, power may be supplied by an external source, e.g., via inductive coupling through the optional antenna 418 or a secondary antenna. The external power source may reside in a device worn on the user's skin or located nearby on a permanent or periodic basis.

If the power source 412 is a rechargeable battery, it can be recharged using the optional antenna 418. In such cases, power can be inductively transferred from an external recharging unit 416 to the battery via the antenna. This setup enables convenient wireless recharging without requiring direct physical connections.

In some embodiments, electrical current can be delivered through electrodes 134 located on a paddle, lead body, or microstimulator housing to stimulate nearby nerve fibers, muscle fibers, or other excitable tissues. The processor 404 typically manages the timing and electrical characteristics of the stimulation. For example, the processor 404 may control parameters such as pulse timing, frequency, amplitude, duration, waveform shape, etc. It can also select which electrodes are active during stimulation and determine their polarity, e.g., assigning specific electrodes as cathodes or anodes. In certain implementations, the processor 404 can further identify electrode configurations that yield optimal stimulation of the target tissue.

The processor may range from a simple electronic device that generates pulses at regular intervals to a more sophisticated unit capable of receiving and interpreting instructions from a programming unit 408 (also referred to as a “programming device” or a “programmer”). For example, such instructions may enable modification of pulse characteristics. In the illustrated embodiment, the processor 404 is connected to a receiver 402, which is further coupled to the optional antenna 418. This configuration allows the processor 404 to receive external commands for controlling parameters such as waveform morphology, pulse attributes, electrode selection, etc., as desired. In some examples, the programming unit 408 can be deemed as part of the electrical stimulation system 400. In some examples, the programming unit 408 can be deemed as external to the electrical stimulation system 400. In some examples, the programming unit 408 can be configured to implement the waveform editing and stimulation parameters programming features, as described more fully below.

In some examples, the antenna 418 can be configured to receive signals (e.g., RF signals) from an external telemetry unit 406, which can be programmed by the programming unit 408. The programming unit 408 may be integrated into or separate from the telemetry unit 406. The telemetry unit 406 can take the form of a wearable device, such as one mounted on the user's skin, or a portable device resembling a pager, cellular phone, or remote control. Alternatively, the telemetry unit 406 may be a stationary device located at a home station or clinician's office. The programming unit 408 may be any suitable system capable of supplying data to the telemetry unit 406 for transmission to the electrical stimulation system 400. This communication may occur via a wired or wireless connection. One example of a suitable programming unit is a computer operated by a clinician or user to send programming commands to the telemetry unit 406.

The signals received by the processor 404 via the antenna 418 and receiver 402 can be used to control or adjust the operation of the electrical stimulation system. These signals may instruct the system to modify stimulation pulse characteristics, including pulse duration, frequency, amplitude, waveform morphology, etc. In addition, the signals may command the system to initiate or cease stimulation, begin or stop battery charging, or perform other control functions. In alternative embodiments, the stimulation system may be configured without an antenna 418 or receiver 402, in which case the processor 404 operates according to pre-programmed parameters.

Optionally, the electrical stimulation system 400 may include a transmitter coupled to the processor 404 and antenna 418 to facilitate outgoing communication to the telemetry unit 406 or another receiving device. For instance, the system may transmit operational status updates, battery charge levels, or notifications of malfunction. The processor 404 may also transmit information about stimulation parameters, allowing the user or clinician to review or verify current settings.

Stimulation energy can be delivered from the control module 102 to the patient's tissue via the lead 103 and its electrodes 134, or alternatively, from a microstimulator through electrodes disposed on the housing of the microstimulator.

Stimulation energy can be delivered in the form of a series of electrical pulses, each characterized by specific properties. These pulses can be defined by stimulation parameters such as waveform morphology, pulse amplitude, pulse frequency (or pulse period), as well as by the selection of one or more electrodes 134 for delivering the stimulation energy. In some embodiments, the pulse amplitude may range from 0 to 25 mA, or from 0 to 50 mA. The pulse frequency may range from 1 Hz to about 15,000 Hz, including narrower ranges such as 1 to 2,000 Hz or 1 to 1,200 Hz, depending on the application.

The stimulation configuration may include one or more electrodes designated as anodes and one or more electrodes designated as cathodes, in any suitable combination. For example, some embodiments can specify a single pulse amplitude and frequency, with specific electrodes identified as either anode or cathode to define the polarity of stimulation. In other embodiments, more complex pulsing schemes may be employed to deliver tailored stimulation patterns. These stimulation pulse schemes can be configured to be charge-balanced (i.e., the net electrical charge delivered during the pulse is approximately zero) to ensure safe and effective delivery of stimulation.

### Example Computing System for Programming Stimulation Waveforms

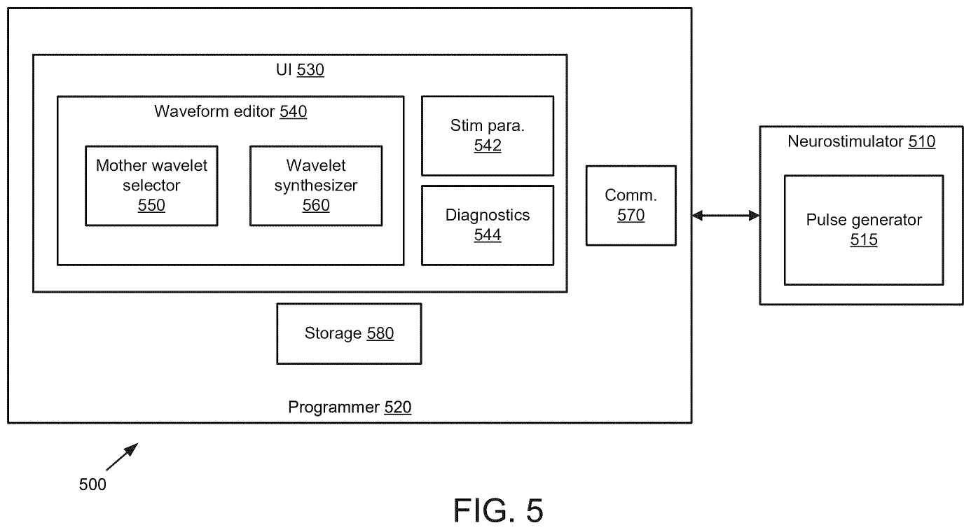

FIG. 5 illustrates an example neuromodulation system 500 that includes a neurostimulator 510 and a programmer 520. The programmer 520 may be an example embodiment of the programming unit 408 described above with respect to FIG. 4, and the neurostimulator 510 may correspond to the remaining components of the electrical stimulation system 400, excluding the programming unit.

The programmer 520 can be configured to communicate with the neurostimulator 510 to program stimulation parameters (e.g., editing or generating a stimulation waveform), retrieve operational or physiological data, and optionally perform system diagnostics or closed-loop control functions. Communication between the programmer 520 and the neurostimulator 510 may be unidirectional or bidirectional and may be implemented via wired or wireless communication protocols.

The programmer 520 includes a user interface (UI) 530, a communication module (Comm.) 570, and a storage module 580. The UI 530 allows a clinician or user to view, create, and/or configure stimulation waveforms for programming the neurostimulator 510. The UI 530 can display waveform representations, parameter settings, and/or system information, and can receive user input through a touchscreen, keyboard, mouse, or other input modality. In the depicted example, the UI 530 includes a waveform editor 540, a stimulation parameter module 542, and a diagnostics module 544. Each of these modules may be executed by one or more processors of the programmer 520 and may interact with one another to facilitate flexible and customizable waveform generation and control.

The waveform editor 540 can be configured to enable user-defined editing of one or more wavelets derived from a mother wavelet to generate a wavelet pulse that defines a morphology of the stimulation waveform. Thus, the generated “wavelet pulse” can also be referred to as the “stimulation waveform.” In some examples, the waveform editor 540 is configured to allow a user to manipulate the shape, timing, and/or spectral content of the stimulation waveform through intuitive graphical or parameter-based controls. In some examples, the waveform editor 540 may include tools or controls for modifying the amplitude, duration, temporal alignment, and/or frequency characteristics of one or more wavelets. In some examples, the waveform editor 540 can be configured to support additional wavelet editing functions, such as applying filters to generate a zero-mean waveform, adding a charge-balancing waveform to compensate for residual charge, etc.

In some examples, the waveform editor 540 can include a mother wavelet selector 550 and a wavelet synthesizer 560 that cooperate to generate and refine a wavelet pulse.

The mother wavelet selector 550 can be configured to allow a user to define or select a mother wavelet from a stored library or to generate a custom mother wavelet. The mother wavelet selector 550 may provide a plurality of selectable mother wavelets representing different waveform families, such as Daubechies, Morlet, or physiologically derived mother wavelets. Physiologically derived mother wavelets can mimic or simulate the waveforms of physiological signals, such as action potentials, evoked compound action potentials (ECAPs), excitatory postsynaptic potentials (EPSPs), inhibitory postsynaptic potentials (IPSPs), among others. In some examples, the mother wavelet selector 550 can further enable user adjustment of one or more descriptors or parameters (e.g., order, scaling, or shape coefficients) that define a characteristic morphology of the selected mother wavelet.

The wavelet synthesizer 560 can be configured to generate one or more wavelets derived from the selected mother wavelet and synthesize a wavelet pulse in a time domain, a time-frequency domain, or a combination thereof. In the time domain, the wavelet synthesizer 560 can modify parameters such as amplitude or temporal characteristics of one or more wavelets, and/or assemble a plurality of wavelets derived from the mother wavelet into a wavelet train or composite waveform. In the time-frequency domain, the wavelet synthesizer 560 can modify a time-frequency representation (e.g., a scalogram) of one or more wavelets and perform an inverse wavelet transform of the modified representation to generate a corresponding time-domain waveform representing the wavelet pulse. The synthesizer 560 may thus support time-domain, time-frequency domain, or hybrid synthesis modes, enabling flexible construction and fine-grained control of waveform morphology. Detailed examples of methods for synthesizing the wavelet pulse in both the time domain and time-frequency domain will be described more fully below.

The stimulation parameter module 542 can be configured to control delivery-level stimulation parameters that determine how the generated wavelet pulse is applied to the target tissue. These parameters can include, among others, a stimulation frequency defining the repetition rate of the wavelet pulse, stimulation duration, and burst or duty-cycle patterns specifying how multiple wavelet pulses are grouped or spaced over time. The stimulation parameter module 542 can also manage electrode configuration settings, such as anode and cathode assignments, multipolar arrangements, and/or current-steering profiles that shape the spatial distribution of the electric field. In some embodiments, the stimulation parameter module 542 can further provide control over session-level parameters, including therapy start and stop conditions, ramp-up or ramp-down profiles, and synchronization with sensing or feedback signals from the neurostimulator 510, etc. Collectively, these parameters can define the overall stimulation protocol by specifying when, where, and under what conditions the wavelet pulse generated by the waveform editor 540 is delivered.

The communication module 570 can be configured to manage data exchange between the programmer 520 and the neurostimulator 510. The communication module 570 may transmit waveform data, stimulation parameters, and/or device control instructions from the programmer 520 to the neurostimulator 510, and may receive data such as stimulation logs, sensed physiological signals, system diagnostics, and/or status information from the neurostimulator 510. Communication may be implemented using wired or wireless telemetry, including, but not limited to, inductive coupling, radio-frequency (RF) communication, near-field communication (NFC), Bluetooth®, or optical links, depending on system configuration and power constraints. In some examples, the communication module 570 can support bidirectional communication, allowing the programmer 520 to update therapy parameters in real time based on feedback data or user input. In some examples, the communication module 570 may also implement data integrity and security protocols, such as encryption, error detection, and/or authentication, to ensure reliable and secure transfer of patient and device information.

The diagnostics module 544 can be configured to monitor, analyze, and display operational and/or physiological data received from the neurostimulator 510 via the communication module 570. In some examples, the diagnostics module 544 may provide real-time and/or historical information related to device performance parameters, such as battery status, electrode impedance, output current and/or voltage levels, telemetry signal strength, etc. In some examples, the diagnostics module 544 can further process sensed physiological signals, such as evoked compound action potentials (ECAPs), local field potentials, electromyographic (EMG) activity, etc., to evaluate therapy effectiveness and/or assist with closed-loop control. The diagnostics module 544 may present this information on the user interface 530 in graphical or numerical form, allowing clinicians or users to perform system checks, identify faults, assess electrode integrity, and/or optimize stimulation parameters. In some examples, the diagnostics module 544 may also log diagnostic events and/or generate alerts or notifications when measured parameters fall outside predefined safety or operational thresholds.

The storage module 580 can include one or more types of memory, such as non-volatile memory (e.g., flash or EEPROM) and volatile memory (e.g., RAM), for storing data, software, and configuration files used by the programmer 520. In some examples, the storage module 580 can maintain a library of mother wavelets that can be used by the mother wavelet selector 550. In some examples, user-generated wavelet trains and/or wavelet pulses created through the waveform editor 540 can be stored in the storage module 580. In some examples, the storage module 580 can store stimulation programs and/or patient-specific therapy profiles defined using the stimulation parameter module 542. In some embodiments, the storage module 580 can store device configuration data, system logs, and/or diagnostic records obtained from the neurostimulator 510. The storage module 580 may also contain software instructions executable by one or more processors of the programmer 520 to perform waveform generation, data analysis, communication, and/or display functions. Stored waveforms and stimulation settings can be retrieved, modified, and retransmitted to the neurostimulator 510 to support repeatable, consistent therapy delivery. In some examples, the storage module 580 may further support encrypted or cloud-synchronized storage, enabling secure backup, remote access, and/or multi-session therapy tracking.

It should be understood that the programmer 520 can include additional components not shown in FIG. 5, such as a processor for executing control algorithms, a display for visualizing waveform morphology or device status, a power supply for portable operation, a network interface for remote monitoring or cloud synchronization, a logging module for storing therapy history and session data, etc.

The neurostimulator 510 can receive the programmed wavelet pulse and corresponding stimulation parameters from the programmer 520 and generate stimulation signals according to the wavelet pulse. The neurostimulator 510 can include a pulse generator 515 for outputting stimulation waveforms corresponding to the programmed wavelet pulse through one or more output channels. Although the term “pulse generator” is used herein for simplicity, it should be understood that the pulse generator 515 can represent any form of tissue stimulation apparatus, including stimulators configured for electrical, ultrasonic, mechanical, or other forms of energy-based stimulation. In such cases, the wavelet pulse generated by the programmer 520 can be converted into corresponding control or drive signals appropriate for delivery by the respective stimulation modality. Accordingly, references to “electrical stimulation” or an “electrical stimulation system” throughout this disclosure should be interpreted broadly to encompass these and other energy-based stimulation systems capable of delivering wavelet-defined stimulation patterns.

In some examples, the neurostimulator 510 may also include control circuitry for executing stimulation commands, and output drivers for delivering the stimulation to one or more electrodes. In some examples, the neurostimulator 510 may further include sensing circuitry to monitor bioelectrical signals and/or physiological responses and may transmit sensed data or feedback information to the programmer 520 to adjust stimulation in real time as part of a closed-loop control algorithm.

In practice, the systems shown herein, such as the neuromodulation system 500, can vary in complexity, with additional functionality, more complex components, and the like. For example, there can be additional functionality within the programmer 520. Additional components can be included to implement security, redundancy, or the like.

The neuromodulation system 500 and any of the other systems described herein can be implemented in conjunction with any of the hardware components described herein, such as the computing systems described below (e.g., processing units, memory, and the like). In any of the examples herein, the mother wavelets, wavelet trains, wavelet pulses, stimulation parameters, diagnostics data, and the like can be stored in one or more computer-readable storage media or computer-readable storage devices. The technologies described herein can be generic to the specifics of operating systems or hardware and can be applied in any variety of environments to take advantage of the described features.

### Example Mother Wavelets

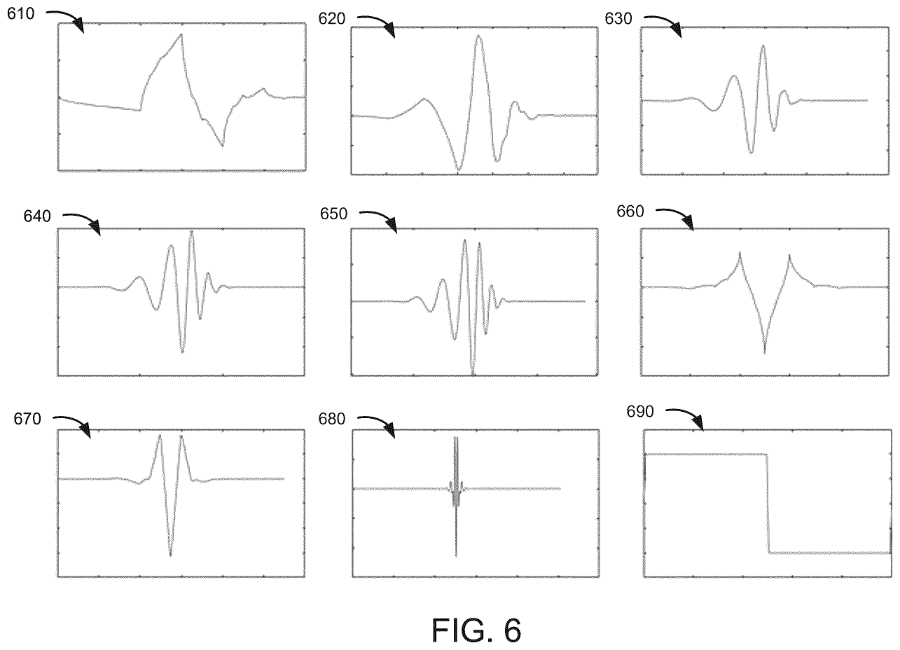

FIG. 6 illustrates a set of example mother wavelets. Each plot in FIG. 6 represents a distinct mother wavelet in the time domain, where the horizontal axis corresponds to time (e.g., in milliseconds) and the vertical axis corresponds to amplitude, which may represent a normalized, dimensionless value or a physical quantity such as current or voltage when scaled for stimulation output. The examples shown include, from top left to bottom right: db2 (610), db4 (620), db6 (630), db8 (640), db10 (650), coif1 (660), coif2 (670), dmey (680), and Haar (690). These mother wavelets are representative of different wavelet families, each exhibiting a characteristic waveform morphology.

A mother wavelet serves as a foundational waveform from which one or more daughter wavelets (or simply wavelets) can be generated through operations such as scaling, shifting, and/or filtering. Each mother wavelet defines a unique waveform morphology that determines how it decomposes or reconstructs signals in time and frequency space. In the context of the present disclosure, the mother wavelet defines the fundamental shape and spectral characteristics of the stimulation waveform. Different wavelet families (e.g., Daubechies, Coiflet, Meyer) exhibit distinct smoothness, symmetry, and compactness properties that can be leveraged to shape stimulation pulses for different therapeutic effects or target tissues.

In the examples shown in FIG. 6, wavelets 610-650 represent Daubechies (db) wavelets of increasing order (db2 through db10). Higher-order Daubechies wavelets generally exhibit greater smoothness, longer temporal support, and more complex oscillatory structures compared to lower-order ones. Similarly, wavelets 660 and 670 correspond to Coiflet (coif1 and coif2) mother wavelets, which are characterized by increased symmetry and a balanced number of vanishing moments for both the scaling and wavelet functions. Wavelet 680 illustrates a Discrete Meyer (dmey) wavelet, which is a smooth, symmetric waveform with a gradual transition in both amplitude and frequency. Wavelet 690 represents the Haar wavelet, which is discontinuous and exhibits a rectangular step-like morphology.

It should be understood that the Haar wavelet (690) is included in FIG. 6 merely for illustrative purposes to contrast discontinuous and continuous waveforms. For purposes of generating the wavelet pulse described herein, Haar wavelets and other discontinuous step-like functions are excluded (e.g., not selectable by the mother wavelet selector 550), because their lack of smooth transitions and discontinuous first derivatives may produce abrupt electrical transitions unsuitable for controlled tissue modulation.

Note that the mother wavelets shown in FIG. 6 exhibit zero-mean oscillations with an amplitude that begins at a baseline value (e.g., zero), increases or decreases, and then returns to the baseline one or more times within a finite duration.

As described herein, the mother wavelets used for generating the wavelet pulse are characterized by having a continuous first derivative and a time-varying instantaneous frequency. The continuous first derivative ensures that the waveform exhibits smooth amplitude transitions without abrupt discontinuities, thereby producing a more physiologically compatible and spectrally controlled stimulation profile. The time-varying instantaneous frequency provides that the waveform's dominant spectral components evolve over time, distinguishing it from periodic sinusoids or fixed-frequency oscillations. This property enables stimulation waveforms that more closely mimic natural bioelectrical signaling patterns and can engage excitable tissues in a more selective and dynamic manner.

Although FIG. 6 illustrates specific examples of mother wavelets, other wavelet families and variants may also be used, including but not limited to Symlet, Biorthogonal, Reverse Biorthogonal, Fejér-Korovkin, and Gaussian (Mexican Hat) wavelets.

Note that in the examples of FIG. 6, wavelets from the same family but of different orders (e.g., db2, db4, db6, db8, db10) are treated as distinct mother wavelets. Alternatively, in some embodiments, all members of a given wavelet family (e.g., all Daubechies wavelets) may be treated collectively as a single mother wavelet, with one or more descriptors, such as order, used to further define its morphology. This hierarchical approach can provide flexibility in how the waveform editor 540 and mother wavelet selector 550 organize and present selectable waveform options, allowing clinicians or users to explore a spectrum of stimulation morphologies across different wavelet families and orders.

### Example Wavelet Pulses

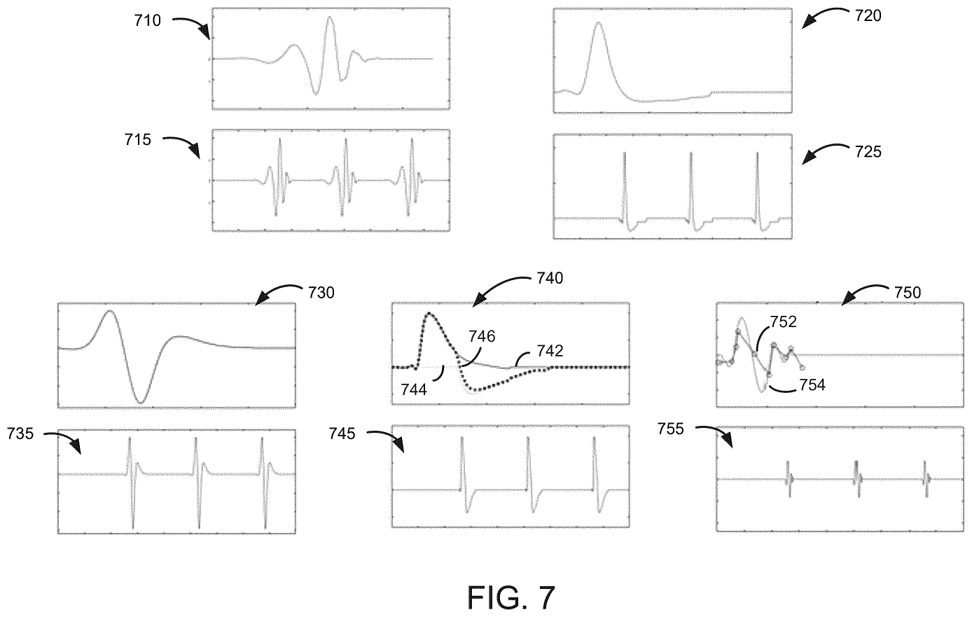

FIG. 7 illustrates several example wavelet pulses and their corresponding mother wavelets that may be used to generate stimulation waveforms according to the techniques described herein. In each plot, the horizontal axis represents time and the vertical axis represents amplitude, which may correspond to a normalized or unitless value or a physical parameter such as current or voltage. Each wavelet pulse shown represents a single unit of a stimulation waveform that can be repetitively delivered at a programmed stimulation frequency to achieve a desired modulation effect.

Waveforms 710 and 715 illustrate an example in which a mother wavelet 710 (e.g., a Daubechies 5 or “db5” wavelet) serves as the basis for generating a wavelet pulse 715. The mother wavelet 710 exhibits a smooth, zero-mean oscillation that begins and ends at a baseline amplitude. In the depicted example, the wavelet pulse 715 comprises three temporally separated wavelets derived from the mother wavelet 710, forming a train-like composite waveform. The db5 wavelet, like many wavelet families (e.g., Daubechies, Coiflets, Meyer, Gaussian, etc.) are inherently zero-mean, which ensures intrinsic charge balance when used for stimulation.

Waveforms 720 and 725 depict an example in which the mother wavelet 720 is based on an ion channel action potential, modeled from a representative biological signal. The mother wavelet 720 includes a characteristic depolarization phase followed by a slower repolarization, approximating the time course of a natural action potential. In the depicted example, the wavelet pulse 725 is composed of three instances of the mother wavelet 720 arranged sequentially in time to form a burst-like stimulation pattern. Although the action potential waveform is approximately zero-mean, minor asymmetries may exist; therefore, in some embodiments, a charge-balancing waveform may be added to ensure net charge neutrality across the wavelet pulse 725.

Waveforms 730 and 735 illustrate a mother wavelet 730 based on an evoked compound action potential (ECAP) morphology and a corresponding wavelet pulse 735 comprising three derived wavelets. The ECAP-based mother wavelet 730 exhibits a broader biphasic response compared to the single-unit action potential waveform, reflecting the synchronized activation of multiple fibers. When used for stimulation, the ECAP-based wavelet pulse 735 can emulate the ensemble electrical dynamics observed in biological systems. Although the ECAP waveform is approximately zero-mean, minor asymmetries may exist. If the wavelet pulse 735 exhibits residual charge imbalance, a compensatory charge-balancing waveform may be applied to maintain overall charge neutrality.

Waveforms 740 and 745 illustrate another physiologically inspired example in which the mother wavelet 740 represents a composite waveform 746 formed from an excitatory postsynaptic potential (EPSP) 742 and an inhibitory postsynaptic potential (IPSP) 744. The composite waveform 746 captures the summative influence of excitation and inhibition in neural signaling. The corresponding wavelet pulse 745, derived from this composite waveform, comprises three sequential wavelets that can be used to model more complex temporal interactions at the synaptic level. As with other physiologically derived mother wavelets, a charge-balancing waveform can optionally be appended if the composite waveform 746 deviates from strict zero-mean symmetry.

Waveforms 750 and 755 show an example of a user-defined mother wavelet 750 and a corresponding wavelet pulse 755 derived therefrom. In this example, a user may specify a waveform shape by plotting or otherwise defining discrete points 752, which can then be smoothed using an interpolation function, such as a spline or polynomial curve, to generate a continuous and smooth waveform 754 suitable for stimulation. In this example, the resulting wavelet pulse 755 includes three wavelets derived from the smoothed user-defined mother wavelet 750.

It should be understood that the examples shown in FIG. 7 are non-exclusive and are provided to illustrate a range of possible waveform morphologies achievable using the disclosed technologies. Variations may include differences in waveform shape, amplitude, temporal width, number of wavelets per pulse, inter-wavelet spacing, and other parameters. Additional mother wavelets may be derived from other physiological signals, such as calcium channel membrane currents or electromyographic signals, as well as from mathematical functions or computationally optimized templates. The generated wavelet pulse may include a single wavelet or a plurality of wavelets, and in some examples, the wavelet pulse can be generated to include multiple trains of wavelets derived from the same or different mother wavelets. In each case, the wavelet pulse defines a finite-duration stimulation unit having a time-varying instantaneous frequency or a defined frequency spectrum that can be repeated or patterned according to programmed stimulation parameters to achieve targeted modulation of electrically excitable tissue.

### Example Methods for Wavelet-Based Electrical Stimulation

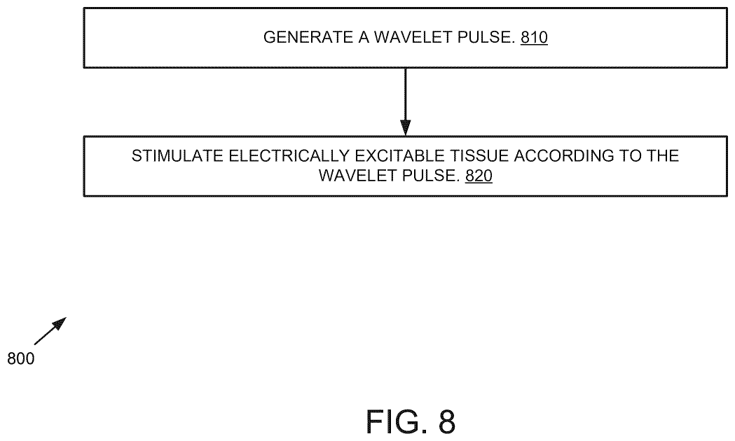

FIG. 8 is a flowchart illustrating an example overall method 800 for wavelet-based electrical stimulation. The method 800 can be performed by the neuromodulation system 500 of FIG. 5.

At step 810, the method 800 can generate, by a programming device (e.g., programmer 520), a wavelet pulse comprising one or more wavelets derived from a mother wavelet. The mother wavelet has a continuous first derivative and a time-varying instantaneous frequency.

At step 820, the method 800 can deliver, by a pulse generator (e.g., pulse generator 515), electrical stimulation to electrically-activatable tissue according to the wavelet pulse.

In some examples, the wavelet pulse can include a plurality of wavelets generated from the mother wavelet and separated in time from one another according to a predefined frequency defining an inter-wavelet duration.

In some examples, the wavelet pulse can include a single wavelet derived from the mother wavelet.

In some examples, the wavelet pulse can include two or more trains of wavelets having different characteristics within a finite duration of the wavelet pulse.

In some examples, the wavelet pulse can include one or more first wavelets and one or more second wavelets derived from the mother wavelet. The one or more second wavelets differ from the one or more first wavelets in at least one of wavelet amplitude, wavelet duration, inter-wavelet duration, or wavelet order.

In some examples, the mother wavelet is a first mother wavelet and the one or more wavelets are first wavelets. The wavelet pulse can further include one or more second wavelets derived from a second mother wavelet different from the first mother wavelet.

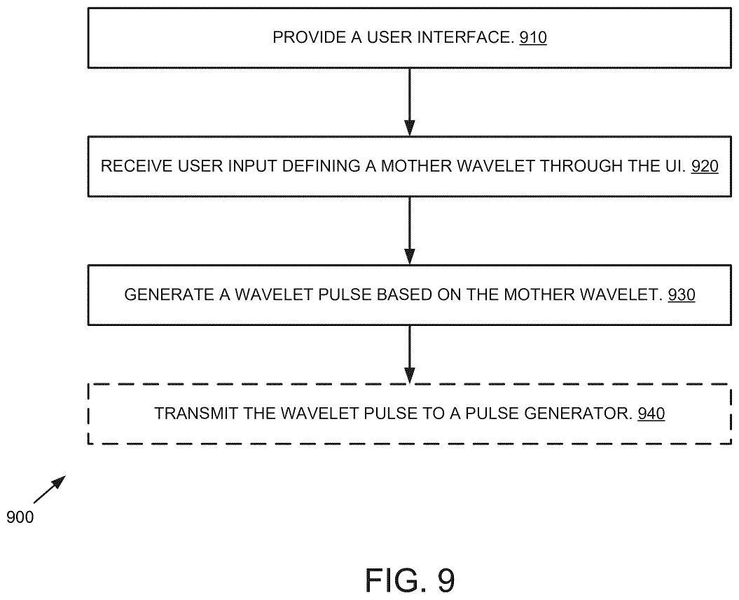

FIG. 9 is a flowchart illustrating an example overall method 900 for programming a stimulation waveform for electrical stimulation. In some examples, the step 810 of FIG. 8 can be implemented by the method 900.

At step 910, the method 900 can provide, by a computing system (e.g., programmer 520), a user interface (e.g., UI 530).

At step 920, the method 900 can receive, through the user interface, user input defining a mother wavelet (e.g., via the mother wavelet selector 550).

At step 930, the method 900 can generate, by the computing system, a wavelet pulse defining a morphology of the stimulation waveform. The wavelet pulse is synthesized (e.g., by the wavelet synthesizer 560) from one or more wavelets derived from the mother wavelet.

Optionally, at step 940, the method 900 can further transmit (e.g., via the communication module 570) the wavelet pulse to a pulse generator (e.g., 515) of the electrical stimulation system configured to electrically modulate electrically-activatable tissue according to the wavelet pulse.

In some examples, the method 900 can further filter the mother wavelet to generate a zero-mean version of the mother wavelet for use in synthesizing the wavelet pulse.

In some examples, the method 900 can add a charge-balancing waveform to the wavelet pulse to compensate for residual charge of the one or more wavelets.

In some examples, generating the wavelet pulse includes synthesizing the wavelet pulse in a time domain by modifying an amplitude or temporal characteristic of the one or more wavelets.

In some examples, generating the wavelet pulse includes synthesizing the wavelet pulse in a time domain by assembling a plurality of wavelets into a composite waveform.

In some examples, generating the wavelet pulse includes synthesizing the wavelet pulse in a time-frequency domain by modifying a time-frequency representation of the one or more wavelets.

In some examples, synthesizing the wavelet pulse in the time-frequency domain further includes performing an inverse wavelet transform of the modified time-frequency representation of the one or more wavelets.

The methods 800, 900, and any of the other methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

The illustrated actions can be described from alternative perspectives while still implementing the technologies. For example, “receive” can also be described as “send” from a different perspective.

### Example User Interface with Waveform Editor

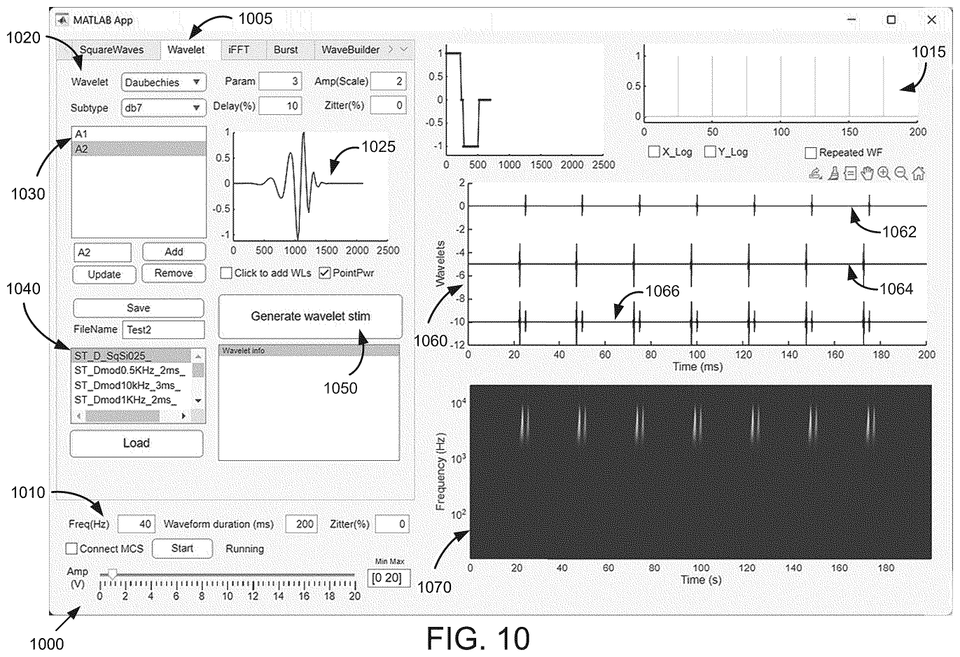

FIG. 10 illustrates an example user interface 1000 implemented as a prototype in MATLAB® for programming stimulation waveforms based on wavelets. The user interface 1000 includes a waveform editor 1005, which provides a visual and interactive environment for defining waveform parameters, selecting mother wavelets, generating wavelet pulses, and transmitting the generated wavelet pulses to a neurostimulator or other electrical stimulation device. The waveform editor 1005 includes multiple interactive controls organized into multiple sections. It should be understood that the arrangement of controls and parameters depicted in FIG. 10 is provided for illustrative purposes only, and in other embodiments, the layout, number, and/or type of controls may vary, and additional functions may be provided.

The waveform editor 1005 is configured to enable wavelet-based stimulation waveform synthesis. The user may define base waveform parameters 1010, which can include frequency, amplitude (or scaling), waveform duration, and optional stochastic variation (e.g., jitter). The waveform duration defines a finite duration of a wavelet pulse, i.e., sets the total time span of a single wavelet pulse (e.g., 200 ms). The amplitude defines the peak magnitude of the individual wavelets (e.g., 1 V) derived from a selected mother wavelet. In this context, the frequency parameter specifies the inter-wavelet duration or spacing within a single wavelet pulse, i.e., how often (and thus how many) wavelets appear within the defined waveform duration. Note that this is distinct from the stimulation frequency (a delivery-level stimulation parameter, e.g., specified by the stimulation parameter module 542), which determines how often wavelet pulses are repeated and delivered to tissue. In one specific example, the waveform duration and frequency can be set so that each wavelet pulse includes a single wavelet.

As shown, a trace 1015 can provide a schematic showing where the individual wavelets occur within the waveform duration and their relative peak amplitudes. The trace 1015 may be displayed on linear or logarithmic axes. As an example, with waveform duration=200 ms and frequency=40 Hz, the wavelet pulse can have seven wavelets that are equally spaced across the 200 ms window. The waveform editor 1005 can dynamically update the trace 1015 as the base waveform parameters 1010 change to provide real-time visualization of the programmed configuration.

As shown, a mother wavelet parameter panel 1020 allows the user to select and configure a mother wavelet from a library of available types (e.g., Daubechies, Coiflet, Morlet, physiologically derived waveforms, etc.). The user can further define one or more waveform descriptors such as subtype (e.g., a particular member within a family such as db7), order (which specifies the mathematical complexity or number of vanishing moments of the wavelet), amplitude scaling, temporal delay (to prevent overlap between traces), and jitter (a controlled stochastic variation in the frequency parameter). A mother wavelet display 1025 can provide a graphical representation of the selected mother wavelet, allowing the user to visualize its morphology prior to synthesis of wavelet pulse.

Once the mother wavelet is selected, the waveform editor 1005 allows the user to synthesize the wavelet pulse in the time domain, the time-frequency domain, or a combination of both. In the time domain, the user can specify (e.g., in a time-domain display 1060) how one or more wavelets derived from the selected mother wavelet are arranged in time to form a train or composite waveform. In the time-frequency domain, the user may manipulate a time-frequency representation (e.g., in a scalogram 1070) based on the selected mother wavelet and apply an inverse wavelet transform to generate the corresponding time-domain waveform (or wavelet pulse).

The inclusion of a jitter parameter allows the frequency that defines the inter-wavelet duration to include a stochastic variation, thereby producing non-uniform temporal spacing between successive wavelets. In some circumstances, this controlled randomness can help prevent neural habituation or entrainment that can occur with strictly periodic stimulation and may promote more natural, desynchronized neural activation patterns. Additionally, jittered timing may help reduce variability in neural response thresholds and minimize adaptation during chronic stimulation, thus improving long-term efficacy and comfort of therapy.

A trace management control panel 1030 allows the user to create, modify, and manage multiple wavelet trains (or traces) that can be combined to form a single wavelet pulse. As illustrated in FIG. 10, the time-domain display 1060 shows an example of two such wavelet trains, including trace 1062 (e.g., trace A1) and trace 1064 (e.g., trace A2), and their combination represented as a composite waveform 1066.

Each trace can be created through a sequence of user-defined actions. For example, the user may define the base waveform parameters 1010 (e.g., duration, amplitude, and frequency) and select a mother wavelet with corresponding descriptors (e.g., subtype, order, scaling) via the mother wavelet parameter panel 1020. These parameters may be configured in any order or iteratively adjusted to achieve the desired waveform characteristics. The system then generates a trace corresponding to a wavelet train derived from the defined parameters. The user can assign a name or label to the generated trace for reference and may create additional traces that differ in waveform morphology, amplitude, frequency, and/or temporal alignment. The trace management control panel 1030 can also be configured to allow the user to add, update, and/or remove traces, providing an intuitive mechanism to organize and combine multiple waveform components that collectively define the structure of the wavelet pulse.

Each wavelet train (e.g., A1, A2) can be composed of wavelets derived from the same or different mother wavelets. For example, wavelet train A1 may be based on a Daubechies-5 mother wavelet, while wavelet train A2 may be based on a Coiflet-2 mother wavelet, thereby producing wavelets of different morphology. Alternatively, both trains may be derived from the same mother wavelet but differ in one or more wavelet parameters, such as amplitude, temporal width, and/or order, yielding distinct waveform characteristics within a common wavelet family. Although FIG. 10 illustrates two wavelet trains, it should be understood that a wavelet pulse may include only a single train, or more than two trains, and each train may include a single wavelet or multiple wavelets depending on the desired stimulation configuration.

The user can also specify frequency parameters that define the inter-wavelet duration for each wavelet train, such that the two trains may have substantially equal inter-wavelet durations or different inter-wavelet durations, producing synchronized or asynchronous patterns, respectively. In addition, the user may adjust temporal delay or offset parameters to shift one wavelet train relative to another in time, allowing the combined waveform (e.g., composite waveform 1066) to exhibit controlled overlap or separation between the trains. Through these configurable relationships (e.g., variation in waveform morphology, amplitude, frequency, and/or timing), the waveform editor 1005 supports generation of complex, multi-train wavelet pulses that can emulate the diversity of natural bioelectrical signaling patterns.

A waveform management panel 1040 allows the user to manage storage and retrieval of generated traces and wavelet pulses. For example, the user can save (e.g., into the storage module 580) individual traces and/or wavelet pulses, including waveform configurations and parameter sets under descriptive names for future access. Saved files may include information such as selected mother wavelet type, associated descriptors, amplitude and frequency parameters, inter-train timing relationships, and/or charge-balance settings. The user can later load previously stored traces and/or wavelet pulses and corresponding configurations to modify, compare, and/or reuse them in subsequent programming sessions. In some examples, the waveform management panel may also support import and export functions, enabling wavelet pulses and waveform configurations to be shared across devices.

The time-domain display 1060 can be configured to present one or multiple wavelet trains (traces) and their combined waveform corresponding to the generated wavelet pulse. As shown in FIG. 10, individual traces such as A1 (1062) and A2 (1064) can be displayed simultaneously along with their composite waveform 1066, providing a clear visualization of how multiple trains interact to form the overall wavelet pulse.

In some implementations, the time-domain display 1060 can support interactive waveform editing, allowing the user to directly manipulate graphical representations of the waveform using a mouse, touch screen, stylus, and/or other input device. For example, the user can adjust the peak amplitude of individual wavelets, modify the temporal spacing or alignment between wavelets, and/or reposition entire wavelet trains within the waveform window by dragging or tapping graphical elements.

The scalogram 1070 provides a time-frequency representation of the generated wavelet pulse, displaying how the frequency content of the wavelet pulse evolves over time. In one example, the horizontal axis corresponds to time, while the vertical axis corresponds to frequency. The color intensity or gray scale shading at each point represents the relative energy or amplitude of the waveform at a given time-frequency coordinate, thus providing a visual map of how spectral power is distributed throughout the duration of wavelet pulse. The scalogram 1070 allows users to assess the dynamic spectral characteristics of the wavelet pulse, including changes in dominant frequency components over time. For a given time-frequency pattern, the waveform editor 1005 can apply an inverse wavelet transform, which may be implemented as a continuous wavelet transform (CWT) or a discrete wavelet transform (DWT), to reconstruct the corresponding time-domain waveform or wavelet pulse.

In some implementations, the scalogram 1070 may also support interactive manipulation, allowing the user to modify the time-frequency content of the wavelet pulse directly. For example, using a mouse, touch screen, stylus, and/or other input device, the user may select specific regions on the scalogram 1070 to increase, decrease, add, and/or remove frequency components at desired time intervals. These user interactions can alter the spectral composition of the waveform, thereby influencing the corresponding time-domain morphology of the wavelet pulse. For example, at a given time point, enhancing lower-frequency regions may broaden or smooth the local wavelet oscillations at that time point, whereas amplifying higher-frequency regions may increase the temporal precision or sharpness of waveform features at the time point.

In some examples, the waveform editor 1005 can maintain real-time synchronization between the time-domain display 1060 and the scalogram 1070, such that changes made in one domain are instantaneously reflected in the other. For example, when the user edits the waveform directly in the time-domain display 1060 (e.g., by adjusting amplitude or timing of individual wavelets), the scalogram 1070 can be automatically recalculated and updated to show the resulting spectral changes. Conversely, when the user modifies the scalogram 1070 by adjusting time-frequency regions, the corresponding time-domain waveform can be immediately regenerated and displayed. Additionally, when the user changes one or more base waveform parameters 1010 and/or parameters in the mother wavelet parameter panel 1020, the time-domain display 1060 and the scalogram 1070 can be automatically and synchronously updated to reflect the new parameter settings.

In some implementations, the waveform editor 1005 can apply a high-pass filter to the selected mother wavelet to generate a zero-mean version of the mother wavelet prior to synthesis of wavelet pulse, thereby removing any baseline offset and ensuring symmetry around the zero-amplitude axis. In cases where the selected mother wavelet is not inherently zero-mean and not high-pass filtered, the waveform editor 1005 can automatically or selectively add a charge-balancing waveform to the generated wavelet pulse to compensate for any residual charge of the one or more wavelets, thereby ensuring overall charge neutrality. Although not expressly depicted in FIG. 10, these operations may be performed automatically in the background or made accessible through additional user controls.

The generate wavelet stimulation control 1050 allows the user to initiate transmission of the generated wavelet pulse to the connected pulse generator. When activated, the waveform editor 1005 can transmit the generated wavelet pulse, including any associated stimulation parameters and charge-balance settings (if any), e.g., through the communication module 570. The pulse generator can then reproduce the transmitted wavelet pulse for delivery to the target tissue or store it for later use. In some implementations, the waveform editor 1005 may be configured to automatically verify waveform integrity and safety limits (e.g., amplitude and/or duration of the wavelet pulse) prior to transmission.

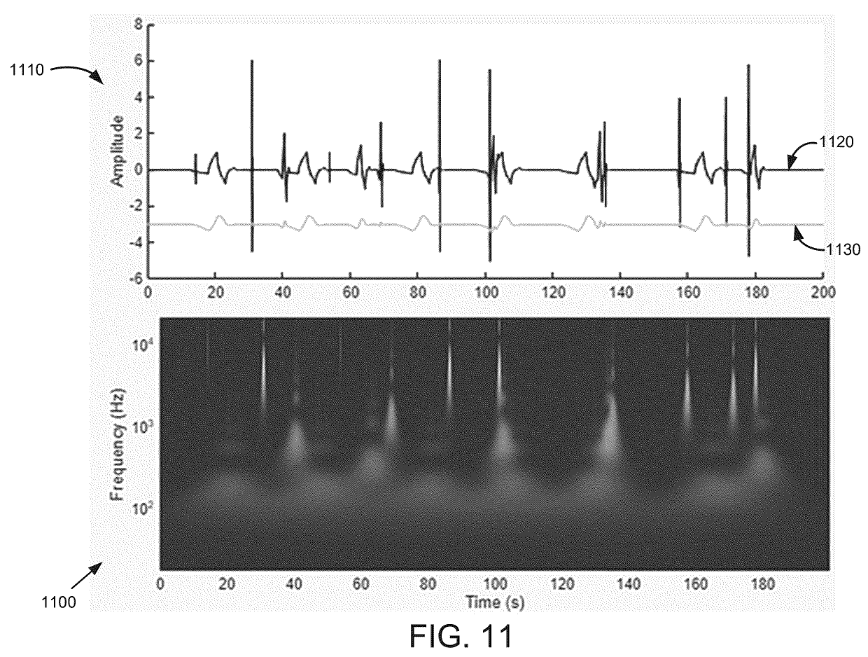

FIG. 11 illustrates another example of a synthesized wavelet pulse generated using the waveform editor, illustrating the ability to design complex wavelet pulse in the time domain, time-frequency domain, or a combination of both. The time-domain plot 1110 shows a representative wavelet pulse 1120 and its corresponding residual charge 1130, which reflects the cumulative charge over time. As shown, the wavelet pulse 1120 includes a plurality of wavelets with varying amplitudes and temporal spacing, forming a complex composite waveform with controlled charge balance. The lower plot (scalogram 1100) displays the time-frequency representation of the wavelet pulse 1120, illustrating the changing frequency content of the wavelet pulse across its duration.

### Example Charge-Balancing

As described above, waveforms or wavelet pulses generated using the disclosed waveform editor can be configured to maintain charge balance, thereby preventing the accumulation of residual charge at the electrode-tissue interface. In some cases, the output waveform (whether composed of a single wavelet or multiple wavelets) may not inherently have a zero-mean value, resulting in a nonzero net charge when integrated over time. If the generated waveform or wavelet pulse (locally or globally) does not satisfy this condition, the waveform editor can implement charge-balancing strategies to correct it, which can be applied automatically or adjusted by the user through selectable controls. On the other hand, if the originally generated wavelet pulse is inherently charge balanced (e.g., having a zero-mean waveform), no additional charge-balancing operation is required.

In some implementations, the waveform editor can provide controls that allow the user to choose among local charge balance, global charge balance, post-wavelet charge recovery balance, and reversal charge balance modes.

In local charge balance, each individual wavelet (or wavelet complex) is constrained such that its net charge delivery is zero, i.e.,

$${\int }_{{t}_{0}}^{{t}_{1}} ψ ( t ) dt = 0$$

where ψ(t) is the instantaneous current and t₀, t₁ bound a single wavelet duration.

In global charge balance, the condition of charge neutrality is applied to the entire wavelet pulse or wavelet train, ensuring

$${\int }_{{t}_{0}}^{{t}_{N}} ψ ( t ) dt = 0$$

where t₀, t_{N} define the duration of wavelet pulse containing N wavelets.

In post-wavelet charge recovery balance, individual wavelets or wavelet trains are permitted to have nonzero residual charge, but the waveform editor can automatically append a charge recovery phase to restore overall charge neutrality. The recovery phase may be implemented as either passive (e.g., via electrode shorting or shunt discharge) or active, using any waveform that accomplishes restoration of charge balance, such as an equal and opposite waveform, a rectangular pulse of appropriate polarity and duration, or another compensatory waveform shape designed to neutralize the accumulated charge. The recovery phase can be appended within the duration of the wavelet pulse (e.g., near the end after all constituent wavelets) or after the wavelet pulse following a predefined interval. In the latter case, the wavelet pulse together with the recovery phase collectively define an updated, charge-balanced wavelet pulse, which forms the stimulation waveform that can be repeated according to a programmed delivery-level stimulation frequency.

In reversal charge balance, a phase-inverted version of the user-defined wavelet or wavelet pulse is delivered subsequent to the original waveform, such that

$${ψ}_{rev} ( t ) = - ψ ( t - Δ t )$$

where Δt is a programmed delay interval between the original and reversed phases.

In some examples, additional charge-phase control parameters may include interphase intervals, defining quiescent pauses between the primary and recovery phases of each waveform.

### Example Feedback Control

In some implementations, the electrical stimulation system disclosed herein can operate in a feedback-controlled mode in which delivery of the wavelet-based stimulation waveform can be dynamically adjusted based on patient-specific feedback signals. For example, during therapy, the stimulation system administers the programmed wavelet stimulation and monitors one or more forms of feedback from the patient, which may include, but not limited to, verbal or vocal responses, physiological signals (e.g., neural or muscular action potentials, electrocardiogram (ECG), respiration rate or depth, blood oxygen saturation, skin temperature, or skin conductivity), and/or motion or activity measurements detected by sensors. The stimulation system can further incorporate data from acoustic, magnetic, and/or imaging modalities, such as ultrasonic reflections indicative of vascular changes and/or magnetic field variations detected through magnetic resonance imaging. These feedback inputs can be processed in real time or intermittently to adjust one or more stimulation parameters (e.g., wavelet amplitude, morphology, frequency, and/or timing, etc.), thereby enabling adaptive, patient-responsive control of the wavelet-based stimulation for optimized therapeutic outcomes.

### Example Experimental Results

Preliminary pilot studies were conducted to explore the feasibility and effects of wavelet-based stimulation for peripheral nerve modulation in human subjects. In these experiments, a custom-built user interface was used to control a multichannel stimulation generator and deliver multi-complex wavelet pulses via adhesive surface electrodes placed over the ulnar nerve. Three Daubechies wavelets (orders 2, 4, and 8) were tested at repetition rates of 5 Hz and 30 Hz, and stimulation thresholds and sensory perceptions were assessed across multiple human participants.

Results from these pilot studies showed that wavelet morphology and repetition frequency influenced both the perception threshold and subjective sensation intensity. Specifically, at lower repetition frequencies (5 Hz), wavelets of higher order (greater complexity) produced higher perceived intensities, whereas at higher frequencies (30 Hz), the trend reversed: higher-order wavelets tended to evoke lower perceived intensities. In one study, a significant reduction in perception threshold was observed at 30 Hz for certain wavelet forms (p<0.05), suggesting that temporal frequency interacts with waveform complexity in modulating neural recruitment. In one pilot study, the spatial distribution of perceived sensation (e.g., within the median nerve field) did not significantly differ between wavelet types at either frequency.

Subjects also reported distinct sensory percepts between wavelet conditions, describing qualitative differences in stimulus “sharpness” and “smoothness,” consistent with changes in morphology of wavelet pulses. These pilot studies demonstrate the feasibility of using wavelets as stimulation primitives and suggest that waveform shape and complexity can be systematically tuned to modulate sensory outcomes. Although preliminary, these results indicate that multi-complex wavelet pulses may offer improved flexibility and selectivity compared to conventional square-wave stimulation, supporting usage of wavelet-based stimulation as a means to refine therapeutic control of neuromodulation.

### Example Advantages

The disclosed wavelet-based stimulation technologies provide several technical advantages over conventional electrical stimulation approaches. By using wavelets, which have finite duration, continuous first derivatives, and time-varying instantaneous frequencies, the disclosed systems enable generation of stimulation waveforms that more closely mimic natural bioelectrical signals, such as action potentials and ECAP. This capability allows for more selective and physiologically relevant activation of electrically excitable tissues, potentially reducing undesired co-activation of non-target neural populations and minimizing patient discomfort. Compared to traditional rectangular or sinusoidal pulses, wavelet-based waveforms can deliver energy more efficiently across a controlled frequency spectrum while maintaining charge balance and producing smoother transitions, which may contribute to more effective stimulation outcomes.

Further, the disclosed waveform editor introduces innovative features that enable highly flexible and precise stimulation waveform design and adjustment. The waveform editor supports real-time synthesis, visualization, and modification of stimulation waveform morphology in both the time domain and time-frequency domain, providing users with intuitive control over the shape and spectral characteristics of the stimulation waveform. Users can modify parameters such as amplitude, duration, inter-wavelet spacing, and frequency content, or combine multiple wavelet trains derived from the same or different mother wavelets to generate customized composite pulses. This flexibility supports rapid iteration and personalization of stimulation patterns tailored to individual physiological responses and/or therapeutic goals. Additionally, the disclosed waveform editor can dynamically update both the time-domain waveform and its scalogram in synchrony, thereby allowing precise temporal and spectral control during waveform synthesis and programming.

Together, the technologies disclosed herein provide a programmable and physiologically inspired stimulation framework that expands the design space for therapeutic neuromodulation and other forms of bioelectrical modulation. Through the integration of fine-grained control over waveform morphology, automatic charge balancing, and real-time visualization and feedback, the disclosed system enables precise tuning of both temporal and spectral characteristics of stimulation waveform. These capabilities allow clinicians and researchers to design and deliver waveforms that are responsive to individual physiological conditions, supporting improved control, reproducibility, and potential therapeutic efficacy in a wide range of stimulation applications.

### Example Computing Systems

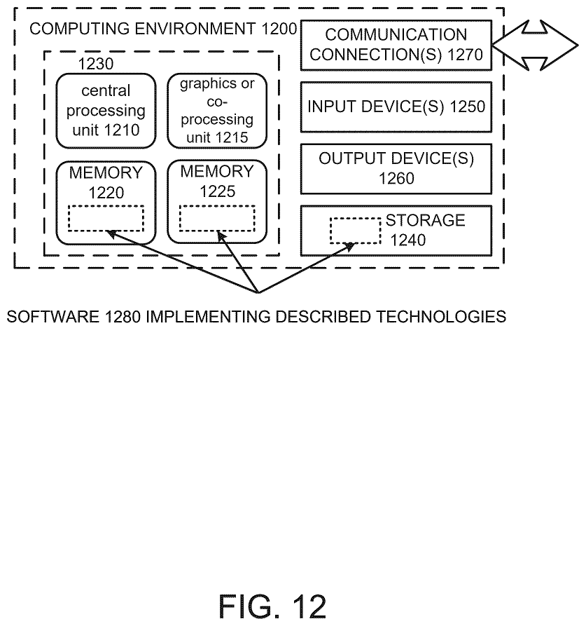

The innovations described herein can be implemented by the computing system 1200 described depicted in FIG. 12. The computing system 1200 is not intended to suggest any limitation as to scope of use or functionality of the present disclosure, as the innovations can be implemented in diverse computing systems.

With reference to FIG. 12, the computing system 1200 includes one or more processing units 1210, 1215 and memory 1220, 1225. In FIG. 12, this basic configuration 1230 is included within a dashed line. The processing units 1210, 1215 can execute computer-executable instructions, such as for implementing the features described in the examples herein. A processing unit can be a general-purpose central processing unit (CPU), processor in an application-specific integrated circuit (ASIC), or any other type of processor. In a multi-processing system, multiple processing units can execute computer-executable instructions to increase processing power. For example, FIG. 12 shows a central processing unit 1210 as well as a graphics processing unit or co-processing unit 1215. The tangible memory 1220, 1225 can be volatile memory (e.g., registers, cache, RAM), non-volatile memory (e.g., ROM, EEPROM, flash memory, etc.), or some combination of the two, accessible by the processing unit(s) 1210, 1215. The memory 1220, 1225 can store software 1280 implementing one or more innovations described herein, in the form of computer-executable instructions suitable for execution by the processing unit(s) 1210, 1215.

The computing system 1200 can have additional features. For example, the computing system 1200 can include storage 1240, one or more input devices 1250, one or more output devices 1260, and one or more communication connections 1270, including input devices, output devices, and communication connections for interacting with a user. An interconnection mechanism (not shown) such as a bus, controller, or network can interconnect the components of the computing system 1200. Typically, operating system software (not shown) can provide an operating environment for other software executing in the computing system 1200, and coordinate activities of the components of the computing system 1200.

The tangible storage 1240 can be removable or non-removable, and can include magnetic disks, magnetic tapes or cassettes, CD-ROMs, DVDs, or any other medium which can be used to store information in a non-transitory way and which can be accessed within the computing system 1200. The storage 1240 can store instructions for the software or method (e.g., the beamforming) implementing one or more innovations described herein.

The input device(s) 1250 can be an input device such as a keyboard, mouse, pen, or trackball, a voice input device, a scanning device, touch device (e.g., touchpad, display, or the like) or another device that provides input to the computing system 1200. The output device(s) 1260 can be a display, printer, speaker, CD-writer, or another device that provides output from the computing system 1200.

The communication connection(s) 1270 can enable communication over a communication medium to another computing entity. The communication medium can convey information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

The innovations can be described in the context of computer-executable instructions, such as those included in program modules, being executed in a computing system on a target real or virtual processor (e.g., which is ultimately executed on one or more hardware processors). Generally, program modules or components include routines, programs, libraries, objects, classes, components, data structures, etc. that perform particular tasks or implement particular abstract data types. The functionality of the program modules can be combined or split between program modules as desired in various embodiments. Computer-executable instructions for program modules can be executed within a local or distributed computing system.

For the sake of presentation, the detailed description uses terms like “determine” and “use” to describe computer operations in a computing system. These terms are high-level descriptions for operations performed by a computer, and should not be confused with acts performed by a human being. The actual computer operations corresponding to these terms vary depending on implementation.

### Computer-Readable Media

Any of the computer-readable media herein can be non-transitory (e.g., volatile memory such as DRAM or SRAM, nonvolatile memory such as magnetic storage, optical storage, or the like) and/or tangible. Any of the storing actions described herein can be implemented by storing in one or more computer-readable media (e.g., computer-readable storage media or other tangible media). Any of the things (e.g., data created and used during implementation) described as stored can be stored in one or more computer-readable media (e.g., computer-readable storage media or other tangible media). Computer-readable media can be limited to implementations not consisting of a signal.

Any of the methods described herein can be implemented by computer-executable instructions in (e.g., stored on, encoded on, or the like) one or more computer-readable media (e.g., computer-readable storage media or other tangible media) or one or more computer-readable storage devices (e.g., memory, magnetic storage, optical storage, or the like). Such instructions can cause a computing device to perform the method. The technologies described herein can be implemented in a variety of programming languages.

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art. Any theories of operation are to facilitate explanation, but the disclosed systems, methods, and apparatus are not limited to such theories of operation.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (e.g., mechanically or chemically) coupled or linked and does not exclude the presence of intermediate elements between the coupled or associated items absent specific contrary language.

Directions and other relative references (e.g., inner, outer, upper, lower, etc.) may be used to facilitate discussion of the drawings and principles herein, but are not intended to be limiting. For example, certain terms may be used such as “inside,” “outside,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

### Example Clauses

Any of the following clauses can be implemented.
Clause 1. A computing system for programming a stimulation waveform for delivery by an electrical stimulation system, the computing system comprising: a user interface comprising a waveform editor configured to enable user-defined editing of one or more wavelets derived from a mother wavelet to generate a wavelet pulse, the wavelet pulse defining a morphology of the stimulation waveform; and a communication interface configured to transmit the wavelet pulse to a pulse generator of the electrical stimulation system configured to electrically modulate electrically-activatable tissue according to the wavelet pulse.
Clause 2. The computing system of clause 1, wherein the waveform editor is further configured to provide a plurality of selectable mother wavelets representing different waveform families from which the one or more wavelets are derived.
Clause 3. The computing system of clause 2, wherein the plurality of selectable mother wavelets includes one or more physiologically derived waveforms representing electrophysiological signals, including at least one of an action potential, evoked compound action potential, excitatory postsynaptic potential, or inhibitory postsynaptic potential (IPSP).
Clause 4. The computing system of any one of clauses 1-3, wherein the waveform editor is further configured to enable user selection of one or more descriptors of the mother wavelet that define a characteristic shape of the mother wavelet.
Clause 5. The computing system of any one of clauses 1-4, wherein the waveform editor is configured to apply a high-pass filter to the mother wavelet to generate a zero-mean version of the mother wavelet for use in generating the wavelet pulse.
Clause 6. The computing system of any one of clauses 1-5, wherein the waveform editor is configured to add a charge-balancing waveform to the wavelet pulse to compensate for residual charge of the one or more wavelets.
Clause 7. The computing system of any one of clauses 1-6, wherein the waveform editor is configured to synthesize the wavelet pulse in a time domain, comprising modifying an amplitude or temporal characteristic of the one or more wavelets.
Clause 8. The computing system of any one of clauses 1-7, wherein the waveform editor is configured to synthesize the wavelet pulse in a time domain, comprising assembling a plurality of wavelets derived from the mother wavelet into a composite waveform.
Clause 9. The computing system of any one of clauses 1-8, wherein the waveform editor is configured to synthesize the wavelet pulse in a time-frequency domain, comprising modifying a time-frequency representation of the one or more wavelets.
Clause 10. The computing system of any one of clauses 1-9, wherein synthesizing the wavelet pulse in the time-frequency domain further comprises performing an inverse wavelet transform of the modified time-frequency representation of the one or more wavelets.
Clause 11. A computer-implemented method for programming a stimulation waveform for delivery by an electrical stimulation system, the method comprising: providing, by a computing system, a user interface; receiving, through the user interface, user input defining a mother wavelet; and generating, by the computing system, a wavelet pulse defining a morphology of the stimulation waveform, wherein the wavelet pulse is synthesized from one or more wavelets derived from the mother wavelet.
Clause 12. The computer-implemented method of clause 11, further comprising transmitting, by the computing system, the wavelet pulse to a pulse generator of the electrical stimulation system configured to electrically modulate electrically-activatable tissue according to the wavelet pulse.
Clause 13. The computer-implemented method of any one of clauses 11-12, further comprising filtering, by the computing system, the mother wavelet to generate a zero-mean version of the mother wavelet for use in synthesizing the wavelet pulse.
Clause 14. The computer-implemented method of any one of clauses 11-13, further comprising adding, by the computing system, a charge-balancing waveform to the wavelet pulse to compensate for residual charge of the one or more wavelets.
Clause 15. The computer-implemented method of any one of clauses 11-14, wherein generating the wavelet pulse comprises synthesizing the wavelet pulse in a time domain by modifying an amplitude or temporal characteristic of the one or more wavelets.
Clause 16. The computer-implemented method of any one of clauses 11-15, wherein generating the wavelet pulse comprises synthesizing the wavelet pulse in a time domain by assembling a plurality of wavelets into a composite waveform.
Clause 17. The computer-implemented method of any one of clauses 11-16, wherein generating the wavelet pulse comprises synthesizing the wavelet pulse in a time-frequency domain by modifying a time-frequency representation of the one or more wavelets.
Clause 18. The computer-implemented method of clause 17, wherein synthesizing the wavelet pulse in the time-frequency domain further comprises performing an inverse wavelet transform of the modified time-frequency representation of the one or more wavelets.
Clause 19. An electrical stimulation system comprising: a programming device comprising a waveform editor configured to generate a wavelet pulse defining a morphology of a finite-length stimulation waveform, the wavelet pulse being synthesized from one or more wavelets derived from a mother wavelet; and a pulse generator in communication with the programming device and configured to electrically modulate electrically-activatable tissue according to the wavelet pulse.
Clause 20. The electrical stimulation system of clause 19, wherein the waveform editor is configured to synthesize the wavelet pulse in a time-frequency domain, comprising: modifying a time-frequency representation of the one or more wavelets; and performing an inverse wavelet transform of the modified time-frequency representation of the one or more wavelets.
Clause 21. An electrical stimulation system comprising: a programming device configured to program a wavelet pulse; and a pulse generator configured to deliver electrical stimulation to electrically-activatable tissue according to the wavelet pulse programmed by the programming device, wherein the wavelet pulse comprises one or more wavelets derived from a mother wavelet, and wherein the mother wavelet has a continuous first derivative and a time-varying instantaneous frequency.
Clause 22. The electrical stimulation system of clause 21, wherein the wavelet pulse comprises a plurality of wavelets generated from the mother wavelet and separated in time from one another within a finite duration of the wavelet pulse.
Clause 23. The electrical stimulation system of clause 22, wherein the plurality of wavelets are generated based on a frequency defining an inter-wavelet duration.
Clause 24. The electrical stimulation system of clause 23, wherein the frequency defining the inter-wavelet duration includes a stochastic variation to produce non-uniform temporal spacing between successive wavelets.
Clause 25. The electrical stimulation system of any one of clauses 21-24, wherein the one or more wavelets are first wavelets, and wherein the wavelet pulse further comprises one or more second wavelets that have a different morphology than the first wavelets.
Clause 26. The electrical stimulation system of clause 25, wherein the one or more second wavelets are derived from the mother wavelet and have a different amplitude, width, or order than the first wavelets.
Clause 27. The electrical stimulation system of clause 25, wherein the one or more second wavelets are derived from a different mother wavelet than the first wavelets.
Clause 28. The electrical stimulation system of any one of clauses 25-27, wherein the one or more second wavelets have an inter-wavelet duration substantially equal to the first wavelets and are temporally offset from the first wavelets.
Clause 29. The electrical stimulation system of any one of clauses 25-27, wherein the one or more second wavelets have a different frequency defining an inter-wavelet duration than the first wavelets.
Clause 30. The electrical stimulation system of clause 21, wherein the wavelet pulse comprises a single wavelet derived from the mother wavelet.
Clause 31. A method for electrical stimulation, comprising: generating, by a programming device, a wavelet pulse comprising one or more wavelets derived from a mother wavelet, the mother wavelet having a continuous first derivative and a time-varying instantaneous frequency; and delivering, by a pulse generator, electrical stimulation to electrically-activatable tissue according to the wavelet pulse.
Clause 32. The method of clause 31, wherein the wavelet pulse comprises a plurality of wavelets generated from the mother wavelet and separated in time from one another according to a predefined frequency defining an inter-wavelet duration.
Clause 33. The method of clause 31, wherein the wavelet pulse comprises a single wavelet derived from the mother wavelet.
Clause 34. The method of any one of clauses 31-33, wherein the wavelet pulse comprises two or more trains of wavelets having different characteristics within a finite duration of the wavelet pulse.
Clause 35. The method of any one of clauses 31-34, wherein the wavelet pulse comprises one or more first wavelets and one or more second wavelets derived from the mother wavelet, wherein the one or more second wavelets differ from the one or more first wavelets in at least one of wavelet amplitude, wavelet duration, inter-wavelet duration, or wavelet order.
Clause 36. The method of any one of clauses 31-35, wherein the mother wavelet is a first mother wavelet and the one or more wavelets are first wavelets, wherein the wavelet pulse further comprises one or more second wavelets derived from a second mother wavelet different from the first mother wavelet.
Clause 37. A programming device for electrical stimulation, comprising: a user interface configured to enable selection of a wavelet pulse comprising one or more wavelets derived from a mother wavelet, the mother wavelet having a continuous first derivative and a time-varying instantaneous frequency; and a communication unit configured to transmit the wavelet pulse to a pulse generator for delivery of electrical stimulation to electrically-activatable tissue according to the wavelet pulse.
Clause 38. The programming device of clause 37, further comprising a wavelet editor configured to modify the wavelet pulse prior to transmitting the wavelet pulse to the pulse generator.
Clause 39. The programming device of clause 38, wherein the wavelet editor is configured to modify at least one of wavelet order, wavelet amplitude, wavelet scaling, or inter-wavelet spacing of the wavelet pulse.
Clause 40. The programming device of any one of clauses 38-39, wherein the wavelet editor is configured to modify a time-frequency representation of the wavelet pulse prior to transmitting the wavelet pulse to the pulse generator.

The technologies from any clause can be combined with the technologies described in any one or more of the other clauses.

In view of the many possible embodiments to which the principles of the disclosed technology can be applied, it should be recognized that the illustrated embodiments are examples of the disclosed technology and should not be taken as a limitation on the scope of the disclosed technology. Rather, the scope of the disclosed technology includes what is covered by the scope and spirit of the following claims.

What is claimed is:

1. A computing system for programming a stimulation waveform for delivery by an electrical stimulation system, the computing system comprising:
a user interface comprising a waveform editor configured to enable user-defined editing of one or more wavelets derived from a mother wavelet to generate a wavelet pulse, the wavelet pulse defining a morphology of the stimulation waveform; and
a communication interface configured to transmit the wavelet pulse to a pulse generator of the electrical stimulation system configured to electrically modulate electrically-activatable tissue according to the wavelet pulse.

2. The computing system of claim 1, wherein the waveform editor is further configured to provide a plurality of selectable mother wavelets representing different waveform families from which the one or more wavelets are derived.

3. The computing system of claim 2, wherein the plurality of selectable mother wavelets includes one or more physiologically derived waveforms representing electrophysiological signals, including at least one of an action potential, evoked compound action potential, excitatory postsynaptic potential, or inhibitory postsynaptic potential (IPSP).

4. The computing system of claim 1, wherein the waveform editor is further configured to enable user selection of one or more descriptors of the mother wavelet that define a characteristic shape of the mother wavelet.

5. The computing system of claim 1, wherein the waveform editor is configured to apply a high-pass filter to the mother wavelet to generate a zero-mean version of the mother wavelet for use in generating the wavelet pulse.

6. The computing system of claim 1, wherein the waveform editor is configured to add a charge-balancing waveform to the wavelet pulse to compensate for residual charge of the one or more wavelets.

7. The computing system of claim 1, wherein the waveform editor is configured to synthesize the wavelet pulse in a time domain, comprising modifying an amplitude or temporal characteristic of the one or more wavelets.

8. The computing system of claim 1, wherein the waveform editor is configured to synthesize the wavelet pulse in a time domain, comprising assembling a plurality of wavelets derived from the mother wavelet into a composite waveform.

9. The computing system of claim 1, wherein the waveform editor is configured to synthesize the wavelet pulse in a time-frequency domain, comprising modifying a time-frequency representation of the one or more wavelets.

10. The computing system of claim 9, wherein synthesizing the wavelet pulse in the time-frequency domain further comprises performing an inverse wavelet transform of the modified time-frequency representation of the one or more wavelets.

11. A computer-implemented method for programming a stimulation waveform for delivery by an electrical stimulation system, the method comprising:
providing, by a computing system, a user interface;
receiving, through the user interface, user input defining a mother wavelet; and
generating, by the computing system, a wavelet pulse defining a morphology of the stimulation waveform, wherein the wavelet pulse is synthesized from one or more wavelets derived from the mother wavelet.

12. The computer-implemented method of claim 11, further comprising transmitting, by the computing system, the wavelet pulse to a pulse generator of the electrical stimulation system configured to electrically modulate electrically-activatable tissue according to the wavelet pulse.

13. The computer-implemented method of claim 11, further comprising filtering, by the computing system, the mother wavelet to generate a zero-mean version of the mother wavelet for use in synthesizing the wavelet pulse.

14. The computer-implemented method of claim 11, further comprising adding, by the computing system, a charge-balancing waveform to the wavelet pulse to compensate for residual charge of the one or more wavelets.

15. The computer-implemented method of claim 11, wherein generating the wavelet pulse comprises synthesizing the wavelet pulse in a time domain by modifying an amplitude or temporal characteristic of the one or more wavelets.

16. The computer-implemented method of claim 11, wherein generating the wavelet pulse comprises synthesizing the wavelet pulse in a time domain by assembling a plurality of wavelets into a composite waveform.

17. The computer-implemented method of claim 11, wherein generating the wavelet pulse comprises synthesizing the wavelet pulse in a time-frequency domain by modifying a time-frequency representation of the one or more wavelets.

18. The computer-implemented method of claim 17, wherein synthesizing the wavelet pulse in the time-frequency domain further comprises performing an inverse wavelet transform of the modified time-frequency representation of the one or more wavelets.

19. An electrical stimulation system comprising:
a programming device comprising a waveform editor configured to generate a wavelet pulse defining a morphology of a finite-length stimulation waveform, the wavelet pulse being synthesized from one or more wavelets derived from a mother wavelet; and
a pulse generator in communication with the programming device and configured to electrically modulate electrically-activatable tissue according to the wavelet pulse.

20. The electrical stimulation system of claim 19, wherein the waveform editor is configured to synthesize the wavelet pulse in a time-frequency domain, comprising:
modifying a time-frequency representation of the one or more wavelets; and
performing an inverse wavelet transform of the modified time-frequency representation of the one or more wavelets.
